(19) United States
(12) Patent Application Publication
(10) Pub. No.: US 2025/0264484 A1
(43) Pub. Date: Aug. 21, 2025

(54) METHODS FOR IDENTIFYING AND TREATING DISEASES

(71) Applicant: Genzyme Corporation, Cambridge, MA (US)

(72) Inventors: Can Kayatekin, Cambridge, MA (US); Jennifer Clarke Matthews, Cambridge, MA (US); Sergio Pablo Sardi, Cambridge, MA (US)

(21) Appl. No.: 18/867,941

(22) PCT Filed: May 23, 2023
(86) PCT No.: PCT/IB2023/055309

§ 371 Date: Nov. 21, 2024

Related U.S. Application Data
(60) Provisional application No. 63/345,242, filed on May 24, 2022.

(30) Foreign Application Priority Data
Sep. 9, 2022 (EP) 22194822.7

(51) Int. Cl. G01N 33/92; G01N 33/50

(52) U.S. Cl. CPC G01N 33/92; G01N 33/5047; G01N 2405/10; G01N 2500/10; G01N 2800/2835; G01N 2800/50; G01N 2800/52

(57) ABSTRACT

Provided are methods for assessing aberrant glycosphingolipid processing, and methods for determining the severity and/or progression of diseases associated therewith. Also provided are methods for assessing the likely therapeutic response of a subject to treatment with agents that can modulate glycosphingolipid processing, as well as therapeutic methods which benefit from assessing the patient in accordance with the disclosure. The methods of the disclosure employ an agent (e.g., conduritol-β epoxide) which can challenge the glycosphingolipid pathway in a cell, followed by monitoring the recovery of the cell, e.g., monitoring the restoration of glycosphingolipid flux in the cell.

This disclosure relates to diagnostic methods for assessing aberrant glycosphingolipid processing, and to methods for determining the severity and/or progression of diseases associated therewith, such as Parkinson's disease. Also provided are methods for assessing the likely therapeutic response of a subject to treatment with agents that can modulate glycosphingolipid processing, and associated treatments.

### SUMMARY

Glycosphingolipids (GSLs) are complex lipids responsible for multiple cellular functions, including plasma and vesicle membrane integrity, signal transduction, and cell-to-cell communication. GSL synthesis and degradation are critical for normal cell function and survival. Impairment of GSL degradation is linked to many lysosomal storage disorders and neurodegenerative diseases.

The final reaction in the breakdown of certain GSLs is mediated by glucocerebrosidase (GCase, also called acid β-glucosidase), a hydrolytic enzyme responsible for the lysosomal degradation of glucosylceramide (GL1). Lysosomal GCase is encoded by the gene GBA. Homozygous and compound heterozygous mutations in GBA cause Gaucher's disease (GD), a lysosomal storage disorder (LSD) characterized by inefficient clearance of GL1 and subsequent clinical manifestations. These patients present with altered glycosphingolipid levels that can be normalized by increasing the glucocerebrosidase activity and/or reducing the synthesis of GL1, as respectively demonstrated by the beneficial effects of enzyme replacement therapy or substrate reduction therapy. GBA mutations, even in a heterozygous state, increase the risk for the development of Parkinson's disease (PD) and related synucleinopathies. Currently, GBA mutations represent the most common genetic risk factor for the development of these diseases. Interestingly, there is evidence for reduced GCase activity in the CNS of sporadic PD patients without a known GBA mutation, suggesting alternate, unknown mechanisms linking the reduction in the hydrolytic activity with disease.

LSDs often present with accumulation of lysosomal substrates due to the severe loss of function of one or more enzymes. For instance, in GD, loss of GCase activity leads to the accumulation of GL1 and its deacylated derivative glucosylsphingosine (lyso-GL1), while treatment with a drug that can reduce levels of these substrates (e.g., venglustat) can be used to treat the disease. Changes in the levels of these lysosomal substrates can serve as important biomarkers for determining the efficacy of therapeutic interventions. Unfortunately, no such biomarker has been identified for PD. Despite observations of a reduction in GCase activity in PD patients, there is little apparent substrate accumulation in accessible biofluids from patients (as evaluated by the Parkinson's Progression Markers Initiative) and even in brain tissue samples collected post-mortem. This is likely because patients suffer from, at most, around a 50% loss of GCase function, which, in the absence of additional factors, is sufficient to process its substrates in most cells and tissues. In these patients, accumulations are likely either restricted to particular cell types, or restricted for brief periods of time. These challenges make it difficult to use conventional plasma biomarkers to assess which patients might benefit from therapeutic intervention.

There are very limited mechanisms to identify individuals carrying normal GBA genotypes who nevertheless have altered GSL metabolism. Patients with GD can be readily identified via reduced GCase enzymatic activity in blood, followed by confirmation of the GBA mutation. Identification of heterozygous patients poses different challenges. Although genetic analysis can routinely identify GBA mutations already established to reduce GCase activity, it can fail to inform the relevance of novel mutations. Biochemical quantification of GCase activity demonstrates additional limitations, as patients with heterozygous GBA mutations present with a large variability in activities that can largely overlap with non-GBA mutation carriers. In addition, the presence of PD patients without GBA mutations who have decreased GCase activity suggest that an alternative detection method is warranted. Similarly, a potentially deleterious increase in the synthesis of GSLs is another mechanism that cannot be uncovered by any of the current methods. In summary, there are multiple mechanisms that can lead to altered GSL flux (increased production and/or decreased degradation) and no appropriate methods exist to identify these aberrations. There is, therefore, a need for new methods for the diagnosis and monitoring of conditions associated with aberrant glycosphingolipid processing.

Based on the data presented herein, the present application is concerned with an alternative way to assess defects in the GSL pathway, e.g., partial reductions in the activity of relevant enzymes, which would not be captured using conventional sphingolipid biomarkers, thereby overcoming deficiencies in conventional methods in diagnosing and monitoring disease conditions. The methods described herein present an acute challenge to the glycosphingolipid (GSL) pathway in cells of a patient and the response of the GSL pathway to that challenge, e.g., the change in GSL flux, is then measured. The way in which the GSL pathway recovers after challenge can be used to indicate the type and severity of the patient's condition. In a general sense, GSL pathway challenge involves making an intervention on the pathway (e.g., upregulating or downregulating one or more enzymes involved in the GSL pathway) which reveals differences between individuals when the recovery from the challenge is monitored. One example of a GSL pathway challenge described herein is the acute restriction of GCase activity in an effort to stress this sphingolipid degradation pathway to the maximum extent possible. Conduritol-β epoxide (CBE) is an irreversible covalent binder and inhibitor of glucocerebrosidase which can be used to acutely restrict GCase activity. When cells or animals are treated with this enzyme, the pre-existing pool of GCase becomes permanently inactivated. The further degradation of rapidly accumulating substrates is, therefore, only possible through newly translated GCase enzyme. This requires the new enzyme molecules to be efficiently synthesized, folded, and trafficked to the lysosome. By measuring the time course and extent of GSL accumulation and restoration following CBE clearance or removal, it is possible to evaluate the combined efficiency of enzyme synthesis, trafficking, and catalytic activity. This method uncovers significant GSL flux aberrations that are otherwise unnoticeable, and it provides an assessment of the GSL pathway more generally which can be used to investigate aberrant GSL processing as well as disease status, progression, and treatment efficacy. Other suitable challenge conditions will be apparent to the skilled reader in the context of the present disclosure.

Accordingly, a first aspect provides a method of assessing a subject for aberrant glycosphingolipid processing, the method comprising treating a cell of the subject to challenge the glycosphingolipid pathway in the cell and monitoring the recovery of at least one glycosphingolipid following the challenge. In embodiments, the challenge comprises contacting the cell with an agent which inhibits an enzyme involved in the glycosphingolipid pathway. In embodiments, the enzyme is glucocerebrosidase. In embodiments, the agent is selective for one enzyme involved in the glycosphingolipid pathway.

The agent may be an irreversible inhibitor of the enzyme, e.g., the inhibitor may bind covalently to the enzyme. In embodiments, the agent is a compound of formula (I):

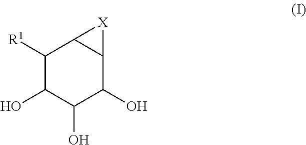

or a pharmaceutically acceptable salt thereof, wherein:
X is —O— or —N(R²)—;
R¹ is selected from —OH, —C(O)OH and —CH₂OH; and
R², when present, is selected from —H and C₁₋₆-alkyl optionally substituted by one or more groups independently selected from —OH and halogen.

The step of treating a cell of the subject to challenge the glycosphingolipid pathway in the cell may comprise contacting the cell with conduritol-ß epoxide (CBE).

In embodiments, the method is an in vitro method in which the cell is present in a sample obtained from the subject. In particular embodiments, the sample is a tissue sample (e.g., from the brain, liver, kidney, skin, or spleen of the subject) or wherein the sample is a cell-containing blood sample (e.g., whole blood).

In embodiments, the cell is a fibroblast, a peripheral blood mononuclear cell (PMBC), or an induced pluripotent stem cell (iPSC) derived from a somatic cell of the subject. In particular embodiments, the cell is a peripheral blood mononuclear cell which has been obtained (e.g., purified) from a blood sample taken from the subject.

In embodiments, the cell expresses neuroepithelial stem cell protein (nestin).

In embodiments, monitoring the recovery of at least one glycosphingolipid following the challenge comprises measuring the level of at least one glycosphingolipid in the cell, or produced by the cell, a plurality of times following the challenge, e.g., 2, 3, 4, 5, 6, 7, 8, 9, 10, or more times. In some embodiments, monitoring the recovery of at least one glycosphingolipid following the challenge comprises measuring the level of at least one glycosphingolipid in the cell, or produced by the cell, for as long as it takes to reach essentially the pre-treatment (baseline) level, e.g., a level which is within about 10% or within about 5% of the pre-treatment level.

In embodiments, the at least one glycosphingolipid is or comprises a lipid selected from glucosylceramide (GL1), ceramide, and glucosylsphingosine (lyso-GL1). In particular embodiments, the at least one glycosphingolipid is or comprises total GL1, total ceramide, or total lyso-GL1. In embodiments, the at least one glycosphingolipid is or comprises a lipid comprising a monounsaturated fatty acid moiety, e.g., selected from C16:1, C18:1, C20:1, C22:1, or C24:1. In embodiments, the at least one glycosphingolipid is or comprises a lipid comprising a fatty acid moiety selected from C24:0, C16:1 n-7, C18:1 n-7, C22:1 n-9, C24:1 n-9, and C18:2 cis. In some embodiments, monitoring the recovery of at least one glycosphingolipid following the challenge does not comprise monitoring the recovery of a lipid comprising a C18:0 fatty acid moiety and/or a lipid comprising a C18:1 fatty acid moiety.

The methods may further comprise a step of comparing the recovery of the at least one glycosphingolipid following the challenge to the recovery of the same glycosphingolipid(s) following the same challenge to a corresponding cell from a healthy individual. In embodiments, the step of comparing involves comparing one or more of the following parameters (which may be derived from a plot of concentration versus time): (i) maximum deviation of the concentration from baseline (Cₘₐₓ); (ii) time taken after challenge to reach Cₘₐₓ (Tₘₐₓ); (iii) area under the curve (AUC); and (iv) time taken for the level to return to baseline, e.g., to return to within about 10% or within about 5% of the baseline level. In such embodiments, the subject may be assessed as having aberrant glycosphingolipid processing if the difference in the parameter(s) is greater than about 10%, e.g., greater than about 20%, 30%, 50%, 75%, 100%, 150%, or 200%.

A second aspect provides a method for determining the severity of a disease associated with aberrant glycosphingolipid processing in a subject, the method comprising assessing the subject for aberrant glycosphingolipid processing in accordance with the first aspect and determining the severity of the disease based on the degree to which the recovery differs from that of a cell from a healthy individual which is challenged in the same way.

In embodiments, the disease is a lysosomal storage disease, e.g., Gaucher's disease. In other embodiments, the disease is a synucleinopathy such as Parkinson's disease (PD), e.g., idiopathic PD.

In some embodiments, the mutational status of the GBA gene in the subject is unknown, or the subject has been assessed as having only one or zero known mutant (e.g., non-functional or reduced function) GBA alleles. In some embodiments, the subject has not previously been diagnosed with a disease associated with aberrant glycosphingolipid processing, e.g., with a lysosomal storage disease such as Gaucher's disease or with Parkinson's disease.

In other embodiments, the subject has previously been diagnosed with a disease associated with aberrant glycosphingolipid processing and the method determines the progression of the disease.

A third aspect provides an in vitro method for diagnosing or monitoring the progression of a synucleinopathy such as Parkinson's disease (PD), e.g., idiopathic PD, in a subject, the method comprising:
obtaining a sample from the subject comprising fibroblast cells, peripheral blood mononuclear cells, or induced pluripotent stem cells derived from somatic cells of the subject, and optionally culturing the cells;
contacting the sample with CBE;
measuring the level of glucosylceramide (GL1) and/or glucosylsphingosine (lyso-GL1) in or produced by the cells a plurality of times to obtain a response curve; and
comparing the response curve with a comparison response curve, wherein the comparison curve is either a standard response curve generated by challenging the same sample obtained from (a) a healthy individual or (b) a patient with a confirmed diagnosis of PD (to make a diagnosis of the subject), or the comparison curve is a response curve from a sample previously obtained from the same subject (to monitor the progression of the disease in the subject).

A fourth aspect provides a method for assessing the likely therapeutic response of a subject to treatment with an inhibitor of glucosylceramide synthase (GCS) or an activator of glucocerebrosidase, the method comprising contacting a cell of the subject with CBE to challenge the glycosphingolipid pathway in the cell and monitoring the recovery of at least one glycosphingolipid following the challenge, whereby the subject is assessed as being a candidate for treatment if the recovery is slower or less complete than the recovery of a cell from a healthy individual which is challenged in the same way. In embodiments, the GCS inhibitor is venglustat or a pharmaceutically acceptable salt thereof. The cell may be a fibroblast, a peripheral blood mononuclear cell, or an induced pluripotent stem cell derived from a somatic cell of the subject. In particular embodiments, the cell is a peripheral blood mononuclear cell which has been obtained (e.g., purified) from a blood sample taken from the subject.

A related aspect provides the use of conduritol-ß epoxide in a method of diagnosing or determining the severity of a disease associated with aberrant glycosphingolipid processing in a subject. In embodiments, the disease is a lysosomal storage (e.g., Gaucher's disease) or a synucleinopathy such as Parkinson's disease (e.g., idiopathic PD).

A fifth aspect provides a method for assessing the therapeutic response of a subject to treatment with an inhibitor of glucosylceramide synthase (GCS) or an activator of glucocerebrosidase (GCase), the method comprising:
(a) obtaining a first cell-containing sample from the subject at a first time point;
(b) contacting a cell of the first cell-containing sample with CBE to challenge the glycosphingolipid pathway in the cell and monitoring the recovery of at least one glycosphingolipid following the challenge;
(c) obtaining a second cell-containing sample from the subject at a second time point after treatment of the subject with the inhibitor of GCS or activator of GCase; and
(d) contacting a cell of the second cell-containing sample with CBE to challenge the glycosphingolipid pathway in the cell and monitoring the recovery of at least one glycosphingolipid following the challenge,
whereby the treatment is assessed as being positive if the recovery in the cell from the second cell-containing sample is faster or more complete than the recovery in the cell from the first cell-containing sample. In embodiments, the treatment of the subject is treatment with the GCS inhibitor venglustat, or a pharmaceutically acceptable salt thereof.

In embodiments, the cell of the first cell-containing sample and/or the cell of the second cell-containing sample is independently selected from a fibroblast, a peripheral blood mononuclear cell, or an induced pluripotent stem cell derived from a somatic cell of the subject.

In some embodiments: the steps (a) and (b) are carried out in essentially the same way as the method steps (c) and (d); the first cell-containing sample and the second cell-containing sample comprise essentially the same cell types; and/or the at least one glycosphingolipid which is monitored in step (b) is the same as the at least one glycosphingolipid which is monitored in step (d).

A sixth aspect provides a method of treating a lysosomal storage disease or a synucleinopathy in a subject in need thereof, the method comprising administering to the subject an agent which is capable of treating the lysosomal storage disease or the synucleinopathy, wherein the method involves monitoring the therapeutic response in accordance with the fifth aspect, and modifying the dose of the agent according to the result of the monitoring. In embodiments, the dose of the agent is kept the same if the treatment is assessed as being positive and the dose of the agent is increased if the treatment is assessed as not being positive; or wherein the dose of the agent is reduced if the treatment is assessed as being positive, with the monitoring steps being continued until the treatment is no longer assessed as being positive, at which time the dosage is increased until the treatment is assessed as being positive again.

A seventh aspect provides a method of treating a lysosomal storage disease or a synucleinopathy in a subject in need thereof, the method comprising the step of administering an effective amount of an agent which is capable of treating the lysosomal storage disease or the synucleinopathy, wherein the subject has been assessed as having aberrant glycosphingolipid processing according to the method of the first aspect or embodiments thereof, or has had their disease state or severity assessed according to the method of the second aspect or the third aspect.

An eighth aspect provides a method of treating or preventing the development or progression of a lysosomal storage disease or a synucleinopathy in a subject assessed as being at risk of developing a lysosomal storage disease or a synucleinopathy according to the method of the second aspect or the third aspect, the method comprising the steps of:
(a) starting the subject on a course of therapeutic treatment; and optionally
(b) assessing or repeating the assessment of risk of developing a lysosomal storage disease according to the method of the second aspect or the third aspect, and optionally adjusting the therapeutic treatment based on the new assessment.

Additional features and advantages of compounds, compositions, and methods disclosed herein will be apparent from the following detailed description.

### BRIEF DESCRIPTION OF THE DRAWINGS

FIG. 1 shows a CBE dose titration study in WT mice that revealed dose-dependent decreases in GCase activity after 24 hours in both cortex (FIG. 1A) and liver (FIG. 1B). N=4 per group (8 for untreated and 100 mg/kg plasma). Columns sharing the same letter were not significantly different from one another. Error bars show SEM.

FIG. 2 shows a CBE dose titration study in WT mice that revealed dose-dependent increases in lipid accumulation after 24 hours. Lyso-GL1 was detected in cortex (FIG. 2A), liver (FIG. 2B), and plasma (FIG. 2C). N=4 per group (8 for untreated and 100 mg/kg plasma). Results for lyso-GL1 are μg/g wet tissue. Columns sharing the same letter were not significantly different from one another. Error bars show SEM.

FIG. 3 shows a CBE timecourse study in Gba^{D409V/D409V}, Gba^{D409V/+}, and Gba^{+/+} mice. This revealed peak GSL accumulation at 24-48 hours with a time-dependent restoration out to 72 hours, having a genotype-phenotype correlation. Following a single intraperitoneal CBE injection, homozygous mice displayed higher peak accumulation of GL1 and lyso-GL1 compared to heterozygous and WT mice in all regions evaluated, namely cortex (FIG. 3A), liver (FIG. 3B), and plasma (FIG. 3C). For each plot, the top line (with squares) is the homozygous D409V mice, the middle line (with triangles pointing upwards) is the heterozygous mice, and the bottom line (triangles pointing downwards) is the wild-type mice. N=10 per group. Data are shown as mean±SEM.

FIG. 4 shows CBE timecourse studies in different WT mouse strains that revealed differences in lipid flux. GSL levels were measured in cortex (FIG. 4A), liver (FIG. 4B), and plasma (FIG. 4C). All mice showed peak glycosphingolipid levels at 24 hours (lyso-GL1, left; total GL1, right). C57BL/6 mice, however, exhibited an increased sensitivity to GCase inhibition, as shown in liver and plasma lyso-GL1 levels and liver GL1 levels. Plasma GL1 levels revealed a different trend, with FVB and C57BL/6 mice exhibiting increased total GL1 levels compared to BALB/cJ mice. No differences were observed in total GL1 levels in cortex. For each plot, the light gray line with circles shows results for BALB/cJ mice, the black line with squares is for C57BL/6 mice, and dark gray line with triangles is for FVB mice. Data are shown as mean±SD.

FIG. 5 shows CBE timecourse studies in different WT mouse strains, measuring levels of different GL1 isoforms in cortex. Each graph reports the amount of the GL1 isoform which was measured: FIG. 5A shows the C22 isoform; FIG. 5B shows the C23 isoform;

FIG. 5C shows the C24 isoform; and FIG. 5D shows the C24:1 monounsaturated isoform. Upon evaluation, chain length was variably distributed between strains, with C57BL/6 mice having higher levels of C22 (FIG. 5A), C23 (FIG. 5B), C24 (FIG. 5C), and monounsaturated C24:1 (FIG. 5D) isoforms. For each plot, the light gray line with circles shows results for BALB/cJ mice, the black line with squares is for C57BL/6 mice, and the dark gray line with triangles is for FVB mice. Data are shown as mean±SD.

FIG. 6 shows the response of human cells to different durations of CBE incubation, namely: 2 hours (black diamonds, top); 24 hours (squares, middle); and 48 hours (gray diamonds, bottom). Data are shown as mean±SD.

FIG. 7 shows the CBE response of human cells which are wild-type (black line with diamonds-100% relative activity when untreated) as compared to GBA knockdown cells (black line with squares-40% reduction in activity) and GBA over-expressing cells (gray line with diamonds-300% increase in activity). Data are shown as mean±SD.

FIG. 8 shows the effect of including sodium taurocholate in the incubation mixture. The response of wild-type cells (dark gray line with diamonds, second from bottom) increases by about a factor of 10 on incubation with sodium taurocholate (black line with squares, top). A similar increase in response is observed for GBA knock-down cells (light gray line with diamonds, bottom) when incubated with sodium taurocholate (light gray with squares, second from top). Data are shown as mean±SD.

FIG. 9 shows the response of human SHSY-5Y neuroblastoma cells to treatment with 100 μM CBE for 24 hours. Total GL1 levels in wild-type cells (FIG. 9A) show no significant difference either with (white bar, right) or without (black bar, left) CBE treatment. Small differences are observed in lyso-GL1 levels (FIG. 9B) after treatment between knock-down (KD), wild-type (WT), and over-expressed (OE) cells. In each case, the lyso-GL1 levels without treatment are very low (left-hand bar, in black). Error bars show SD, and data are presented in relative (arbitrary) units, “AU”, which are normalized for cell number.

FIG. 10 shows the relative accumulation of different GL1 isoforms in human cells after treatment with CBE. The GL1 species are ranked by fatty acid chain length (C14 to C26) with the saturated species to the left and the mono-unsaturated species to the right. Error bars show SD.

FIG. 11 shows the baseline abundance of GL1 isoforms in human cells. As for FIG. 10, the GL1 species are ranked by fatty acid chain length (C14 to C26) with the saturated species to the left and the non-unsaturated species to the right. Error bars show SD, and data are presented in relative (arbitrary) units, “AU”, which are normalized for cell number.

FIG. 12 shows GSL levels in human PBMCs up to 24 hours after CBE challenge (gray circles) as compared with untreated controls (black squares). Lipid levels were normalized to PC concentration in the sample. Levels of GL-1 (FIG. 12A) and lyso-GL1 (FIG. 12B) were markedly increased after challenge and returned to baseline by 16 hours. Data are shown as mean±SEM.

FIG. 13 shows the accumulation of GL1 isoforms in untreated cells over 72 hours. Species are denoted by their fatty acid carbon chain length and saturation (fully saturated unless specified otherwise, e.g., for C24:1). For each isoform, the columns from left to right are baseline (black), 24 hours (gray), 48 hours (dotted), and 72 hours (striped). Data are shown as mean±SEM.

### DETAILED DESCRIPTION

Although specific embodiments of the present disclosure will now be described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology, and recombinant DNA, which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, molecular weight, etc., including ranges, are approximations which are varied (+) or (−) by increments of e.g., 0.1 or 1.0, where appropriate. It is to be understood, although not always explicitly stated, that all numerical designations are preceded by the term “about”, which is used to denote a conventional level of variability. For example, a numerical designation which is “about” a given value may vary by ±10% of said value; alternatively, the variation may be ±5%, ±2%, or ±1% of the value. It also is to be understood, although not always explicitly stated, that the reagents described herein are merely exemplary and that equivalents of such are known in the art.

As used in the specification and claims, the singular form “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, but do not exclude others. “Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass both “consisting essentially of” and “consisting of”.

A “subject,” “individual”, or “patient” is used interchangeably herein, and refers to a vertebrate, such as a mammal. Mammals include, but are not limited to, murines, rats, rabbit, simians, bovines, ovine, porcine, canines, felines, farm animals, sport animals, pets, equines, primates, and humans. In embodiments, the mammals include horses, dogs, and cats. In embodiments, the mammal is a human.

The term “healthy individual” as used herein typically denotes an individual who does not suffer from a synucleinopathy, and/or who does not have any GBA mutations. A healthy individual may lack mutations in any gene which encodes an enzyme involved in the glycosphingolipid pathway, for example mutations in the genes encoding ceramide synthase, glucosylceramide synthase, galactosylceramide synthase, lactosylceramide synthase, sphingomyelin synthase, ceramidase, glucocerebrosidase, saposin, galactosylceramide β-galactosidase, acid sphingomyelinase, arylsulphatase A, α-galactosidase A, β-hexosaminidase (e.g., Hex A or Hex B), sialidase, GM1-β-galactosidase, GM2 ganglioside activator protein, glucosyl transferase, and galactosyl transferase.

“Administering” is defined herein as a means of providing an agent or a composition containing the agent to a subject in a manner that results in the agent being inside the subject's body. Such an administration can be by any route including, without limitation, oral, transdermal (e.g., vagina, rectum, oral mucosa), by injection (e.g., subcutaneous, intravenous, parenterally, intraperitoneally, into the CNS), or by inhalation (e.g., oral or nasal). Pharmaceutical preparations are, of course, given by forms suitable for each administration route.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; and/or (3) relieving the disease, i.e., causing regression of the disease or its clinical symptoms.

The term “suffering” as it relates to the term “treatment” refers to a patient or individual who has been diagnosed with or is predisposed to the disease. A patient may also be referred to being “at risk of suffering” from a disease because of, e.g., a history of disease in their family lineage or because of the presence of genetic mutations associated with the disease. A patient at risk of a disease has not yet developed all or some of the characteristic pathologies of the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents for any particular subject depends upon a variety of factors including, for example, the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. These considerations, as well as effective formulations and administration procedures, are well known in the art and are described in standard textbooks. Consistent with this definition, as used herein, the term “therapeutically effective amount” is an amount sufficient to treat (e.g., improve) one or more symptoms associated with a neurodegenerative condition. For example, oral administration may require a total daily dose of from 0.1 mg to 1000 mg. The total daily dose may be administered in single or divided doses and may, at the physician's discretion, fall outside of the typical range given herein.

As used herein, the term “pharmaceutically acceptable excipient” encompasses any of the standard pharmaceutical excipients, including carriers such as a phosphate buffered saline solution, water, and emulsions, such as an oil/water or water/oil emulsion, and various types of wetting agents. Pharmaceutical compositions also can include stabilizers and preservatives. For examples of carriers, stabilizers, and adjuvants, see Remington's Pharmaceutical Sciences (20th ed., Mack Publishing Co. 2000).

As used herein, the term “pharmaceutically acceptable salt” means a pharmaceutically acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.

Addition salts can be readily prepared using conventional techniques, e.g., by treating a base compound with a defined amount of the chosen mineral or organic acid in an aqueous solvent medium or in a suitable organic solvent such as, for example, methanol or ethanol. Compounds that are positively charged, e.g., containing a quaternary ammonium, may also form salts with the anionic component of various inorganic and/or organic acids. Acids which can be used to prepare pharmaceutically acceptable acid addition salts are those which can form non-toxic acid addition salts, e.g., salts containing pharmacologically acceptable anions, such as chloride, bromide, iodide, nitrate, sulfate or bisulfate, phosphate or acid phosphate, acetate, lactate, citrate or acid citrate, tartrate or bitartrate, succinate, malate, maleate, fumarate, gluconate, saccharate, benzoate, methanesulfonate, and pamoate [i.e., 1,1′-methylene-bis-(2-hydroxy-3-naphthoate)] salts. Bases which can be used to prepare the pharmaceutically acceptable base addition salts are those which can form non-toxic base addition salts, e.g., salts containing pharmacologically acceptable cations, such as, alkali metal cations (e.g., potassium and sodium), alkaline earth metal cations (e.g., calcium and magnesium), ammonium or other water-soluble amine addition salts such as N-methylglucamine (meglumine), lower alkanolammonium, and other such bases of organic amines.

Any reference herein to a compound is to be considered to include reference to the pharmaceutically acceptable salts thereof, although not explicitly stated. Thus, reference to “venglustat” includes pharmaceutically acceptable salts of venglustat, e.g., venglustat malate. Similarly, reference to “eliglustat” includes pharmaceutically acceptable salts of eliglustat, e.g., eliglustat hemitartrate. Venglustat is (S)-1-azabicyclo[2.2.2]octan-3-yl-N-[2-[2-(4-fluorophenyl)-1,3-thiazol-4-yl]propan-2-yl]carbamate. Eliglustat is N-[(1R,2R)-1-(2,3-dihydro-1,4-benzodioxin-6-yl)-1-hydroxy-3-pyrrolidin-1-ylpropan-2-yl] octanamide.

The recitation of an embodiment for a variable or aspect herein includes that embodiment as any single embodiment or in combination with any other embodiments or portions thereof.

The following abbreviations are used herein:
AD Alzheimer's Disease
AUC area under the curve (pharmacokinetics)
CBE conduritol-β epoxide
Cmax maximum concentration (pharmacokinetics)
CNS central nervous system
DLB dementia with Lewy bodies
DNA deoxyribonucleic acid
EC₅₀ half maximal effective concentration
ERT enzyme replacement therapy
FD Fabry disease
GBA gene encoding GCase
GCase glucocerebrosidase (also called acid β-glucosidase)
GCS glucosylceramide synthase
GD Gaucher disease
GL1 glucosylceramide
GL2 lactosylceramide
GL3 globotriaosylceramide
GM1 monosialotetrahexosylganglioside
GM2 monosialotrihexosylganglioside
GM3 monosialodihexosylganglioside
GSL glycosphingolipid
Hex β-hexosaminidase
IC₅₀ half maximal inhibitory concentration
iPSC induced pluripotent stem cells
KD knock-down
lyso-GL1 glucosylsphingosine
lyso-GL2 lactosylsphingosine
lyso-GL3 globotriaosylsphingosine
LSD lysosomal storage disorder
MPS mucopolysaccharidosis
NCL neuronal ceroid lipofuscinosis
OE over-expressed
PBMC peripheral blood mononuclear cell
PCR polymerase chain reaction
PC phosphatidylcholine
PD Parkinson's Disease
RNA ribonucleic acid
SAP saposin
SD standard deviation
SEM standard error of the mean
SRT substrate reduction therapy
Tmax time at maximum concentration (pharmacokinetics)
WT wild-type
   Methods which Challenge GSL Processing in a Cell

As explained in more detail in the following Examples, the present disclosure is directed to a novel assay that can evaluate lipid flux in cells over a short period of time via perturbation of the sphingolipid pathway, e.g., at the GCase node. The assay which is exemplified herein uses conduritol-β epoxide (CBE), an irreversible covalent binder and inhibitor of glucocerebrosidase, which, when administered e.g., intra-peritoneally, inhibits GCase and allows for the measurement of subsequent lipid accumulation and restoration, or flux, of lipids (e.g., GL1 and/or lyso-GL1). Using a GCase mouse model under three genotypic conditions—homogeneous knockout (Gba^{-/-}), heterogenous knockout (Gba^{-/+}), and wild-type (Gba^{+/+})—patterns of lipid flux have been observed which correlate with CBE dosage and with genotype. It has also been observed that wild-type mice of different genetic backgrounds display variable lipid flux dependent on their genotype. These lipid flux patterns were recapitulated in vitro, using human SHSY-5Y neuroblastoma cell lines that were genetically modified to either overexpress GBA or have decreased GCase activity due to GBA knockdown. Lipid levels in response to CBE treatment were shown to correlate inversely with GBA levels.

Emerging evidence suggests that idiopathic, non-GBA mutation carrier patients may exhibit reductions in GCase activity and/or increased glycosphingolipid accumulation in the central nervous system (CNS). This highlights the potential for expanding the target population for substrate reduction therapy (using drugs which modulate the GSL pathway, such as venglustat) based on interrelated cellular and biochemical mechanisms. The results presented herein suggest that uncovering GSL flux impairment by a “challenge” assay (e.g., using CBE or the like) has the potential to expand the use of therapies targeting this pathway to a broader population of patients. The data generated in vivo in mice and in vitro in human cell lines can be extrapolated to the clinical setting, and the present disclosure describes how patient cells, such as peripheral blood mononuclear cells (PBMCs) and/or fibroblasts, could be collected, cultured, and treated to reveal susceptibility to GSL pathway perturbation. This, in turn, could help to expand the patient population that would benefit from venglustat and related therapies.

Broadly speaking, the present disclosure provides methods in which the GSL pathway in a cell from a subject is challenged and the response of the pathway to that challenge (e.g., its recovery) is monitored. This can be used to assess the subject for aberrant glycosphingolipid processing (or to determine the severity of a disease associated therewith), or to diagnose or monitor the progression of a disease associated therewith, or to assess the likely or actual therapeutic response of the subject to treatment with a drug that can modulate the GSL pathway.

Thus, in a first aspect the disclosure provides a method of assessing a subject for aberrant glycosphingolipid processing, the method comprising treating a cell of the subject to challenge the glycosphingolipid pathway in the cell and monitoring the recovery of at least one glycosphingolipid following the challenge.

Typically, the challenge presented to the cell involves contacting the cell with an agent which modulates (e.g., inhibits) an enzyme involved in the glycosphingolipid pathway. Examples of enzymes which may be targeted include: (a) lipid synthases, for example ceramide synthase, glucosylceramide synthase, galactosylceramide synthase, lactosylceramide synthase, and sphingomyelin synthase; (b) lipid hydrolases, for example ceramidase, glucocerebrosidase, saposin (e.g., Sap A, B, C, or D), galactosylceramide β-galactosidase, acid sphingomyelinase, arylsulphatase A, α-galactosidase A, β-hexosaminidase (e.g., Hex A or Hex B), sialidase, GM1-β-galactosidase, and GM2 ganglioside activator protein; and (c) glycosyl transferases, for example glucosyl transferase, and galactosyl transferase. In embodiments, the agent activates a lipid synthase enzyme. In other embodiments, the agent activates a glycosyl transferase enzyme. In other embodiments, the agent inhibits a lipid hydrolase. In one embodiment the enzyme is glucocerebrosidase. In one embodiment, the enzyme is glucocerebrosidase and the agent is an inhibitor of glucocerebrosidase.

In embodiments, the agent is selective for one enzyme involved in the glycosphingolipid pathway. For example, the agent is a factor of at least 10 times more active (as measured, e.g., by IC₅₀ or EC₅₀ value) against the one enzyme than the other enzymes involved in the glycosphingolipid pathway. In embodiments, the agent is a factor of 15, 20, 25, 30, 50, 75, 100, 150, 200, 500, or 1000 more active against the one enzyme than the other enzymes involved in the glycosphingolipid pathway.

In embodiments, the agent is an irreversible modulator of the enzyme involved in the glycosphingolipid pathway. For example, the agent may bind covalently to the enzyme, e.g., to block an active site of the enzyme or to block an interaction between the enzyme and another biological molecule needed for its activity. Alternatively, the agent may target the enzyme for deactivation or degradation. In embodiments, the agent is an irreversible inhibitor of the enzyme involved in the glycosphingolipid pathway. In one embodiment, the agent is an irreversible inhibitor which binds covalently to the enzyme.

Agents which bind strongly (e.g., covalently) to enzymes involved in the glycosphingolipid pathway are known to the skilled person (see, e.g., Canals et al., Br J Pharmacol. (2011) 163 (4): 694-712 for details of agents targeting sphingomyelinases and ceramidases). Such agents are often structural analogues of the natural substrate(s) for the enzyme, for example mimicking the transition state of a native reaction intermediate and/or containing a reactive chemical group which can form a covalent bond with the enzyme. Exemplary covalent modulators include the small molecule inhibitors of glucocerebrosidase described by Kuo et al. (The FEBS Journal (2019) 286:584-600). These compounds are transition state analogues which contain an epoxide group that traps a carboxylic acid on the enzyme. Among the inhibitors described by Kuo et al. is conduritol-β epoxide (CBE), which is highly selective for glucocerebrosidase over other glycosidases (having an IC₅₀ value of around 0.3 μM against GCase, as compared to values in the range of 200-2000 μM against non-lysosomal glucosylceramidase, β-glucuronidase, and α-glucosidases).

Thus, in embodiments the agent is a compound of formula (I):

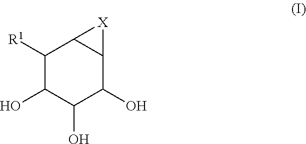

or a pharmaceutically acceptable salt thereof, wherein:
X is —O— or —N(R²)—;
R¹ is selected from —OH, —C(O)OH and —CH₂OH; and
R², when present, is selected from —H and C₁₋₆-alkyl optionally substituted by one or more groups independently selected from —OH and halogen.

In one embodiment, the agent is CBE. Thus, viewed from this aspect the disclosure provides a method of assessing a subject for aberrant glycosphingolipid processing, the method comprising contacting a cell of the subject with CBE to challenge the glycosphingolipid pathway in the cell and monitoring the recovery of at least one glycosphingolipid following said challenge.

In practice, the method will typically be performed on a sample which has been obtained from the subject. Thus, in embodiments the method is an in vitro method in which the cell is present in a sample obtained from the subject. The method may include a step of obtaining a sample from the subject for direct analysis. Alternatively, the method can be carried out on a sample which was previously obtained from the subject and which may, for example, have been stored and optionally reconstituted (e.g., in the case of a dried blood sample or a biopsy sample frozen using liquid nitrogen). The sample may be (or may have been) processed to enrich or concentrate the cells which are to be challenged. Alternatively, or in addition, the sample may be (or may have been) processed to remove unwanted sample material, e.g., tissue, such as connective tissue.

The methods of the disclosure can employ tissue samples, for example samples of tissue from the brain, liver, kidney, skin, or spleen of the subject. The methods of the disclosure can also employ samples of cell-containing biological liquids, for example whole blood or cell-containing fractions thereof. Methods for obtaining such samples are known to the skilled person. Thus, in embodiments the sample is a tissue sample (e.g., from the brain, liver, kidney, skin, or spleen of the subject) or the sample is a cell-containing blood sample (e.g., whole blood). In particular embodiments, the tissue sample is a brain, liver, kidney, skin, or spleen sample. In other particular embodiments, the blood sample is whole blood. Whole blood and cell-containing fractions thereof are especially convenient for use in accordance with the present methods.

In embodiments, the method treats a cell of a particular type (e.g., lineage). Thus, the methods of the disclosure can employ a sample which comprises that cell type, or they can employ purified, passaged, and/or expanded cell populations which may consist essentially of that cell type. The use of partially or fully purified cells and cell populations can improve the sensitivity of the present methods. Examples of cell types which may be used in accordance with this disclosure include fibroblasts, peripheral blood mononuclear cells (PMBCs), and induced pluripotent stem cells (iPSCs). Methods for obtaining, purifying, passaging, and/or expanding such cells are known to the skilled person. It will be understood that methods described herein as being carried out on “a cell” encompass methods in which a plurality of such cells are used, optionally wherein other cells or cell types may also be present.

Thus, in one embodiment the cell is a fibroblast; in a related embodiment the sample comprises (or consists essentially of) fibroblasts. Fibroblasts may be obtained, for example, from dermal tissue of the subject, e.g., from a skin punch biopsy, using conventional techniques. In another embodiment the cell is an iPSC; in a related embodiment the sample comprises (or consists essentially of) iPSCs. iPSCs are generated from somatic cells of the patient, for example by reprogramming of fibroblasts which may be obtained as described herein. Other sources of somatic cells for the generation of iPSCs include keratinocytes (which may be obtained, e.g., from hair follicles), peripheral blood cells, and renal epithelial cells (which may be obtained, e.g., from urine). In another embodiment the cell is a PBMC which has been obtained (e.g., purified) from a blood sample taken from the subject; in a related embodiment the sample comprises (or consists essentially of) PBMCs which have been obtained (e.g., purified) from a blood sample taken from the subject. In embodiments, the cell expresses neuroepithelial stem cell protein (nestin).

The present methods involve monitoring the recovery of the GSL pathway after challenge. Based on the disclosure herein, the skilled reader would appreciate that there are different ways in which this monitoring could be achieved and that the most suitable way (or ways) might be different in different situations, e.g., where different agents are used to challenge different parts of the GSL pathway and/or where different sample or cell types are employed. The discussion below focusses on the situation that is exemplified hereinafter, namely monitoring GSL recovery after treatment of cells with CBE to challenge the GCase node specifically. If other parts of the GSL pathway were challenged, modifications could (if necessary) be made to this assessment on the basis of the present disclosure.

In accordance with the first aspect of the disclosure, the monitoring of recovery of the GSL pathway to challenge is achieved by monitoring the recovery of at least one glycosphingolipid. In embodiments where the challenge involves contacting the cell with an agent that modulates (e.g., inhibits) an enzyme involved in the glycosphingolipid pathway, the at least one glycosphingolipid which is monitored comprises one or more of: (i) a substrate of said enzyme; (ii) an upstream precursor to said substrate of said enzyme; (iii) a product of said enzyme; and (iv) a downstream metabolite of said product of said enzyme.

Suitable glycosphingolipids which can be used for monitoring recovery include GL1, lyso-GL1, GL2, lyso-GL2, GL3, lyso-GL3, galactocerebroside, GM1, GM2, and GM3, as well as, e.g., ceramide and sphingosine. In embodiments, the at least one glycosphingolipid is (or comprises) a lipid selected from glucosylceramide (GL1), ceramide, and glucosylsphingosine (lyso-GL1). In embodiments, the at least one glycosphingolipid is (or comprises) total GL1, total ceramide, or total lyso-GL1. These embodiments are particularly useful in cases where the agent is an inhibitor of GCase. In other embodiments, the at least one glycosphingolipid is (or comprises) total GL3, total ceramide, or total lyso-GL3.

In embodiments, the at least one glycosphingolipid is (or comprises) a lipid (e.g., GL1) comprising a fatty acid moiety which has a low baseline abundance, i.e., a low abundance before any challenge has been presented to the GSL pathway. Such fatty acids may be found at a level which is less than 25% of the total fatty acids on the lipid in question (on a molar basis), for example at a level which is less than 20%, less than 15%, less than 10%, less than 8%, less than 5%, less than 2%, or less than 1% of the total fatty acids on the lipid in question.

In embodiments, the at least one glycosphingolipid is (or comprises) a lipid (e.g., GL1) comprising a monounsaturated fatty acid moiety. For example, the fatty acid moiety may be selected from C16:1, C18:1, C20:1, C22:1, C23:1, C24:1, or C26:1. In embodiments, the fatty acid moiety is selected from C16:1, C18:1, C20:1, C22:1, and C24:1. For example, the fatty acid moiety may be selected from C16:1, C18:1, and C20:1, or from C16:1 and C18:1. In one embodiment, the at least one glycosphingolipid is (or comprises) a lipid comprising a C16:1 fatty acid moiety. In other embodiments, the at least one glycosphingolipid is (or comprises) a lipid comprising a fatty acid moiety selected from C24:0, C16:1 n-7, C18:1 n-7, C22:1 n-9, C24:1 n-9, and C18:2 cis. In embodiments, monitoring the recovery of at least one glycosphingolipid following the challenge does not comprise monitoring the recovery of a lipid (e.g., GL1) comprising a C18:0 fatty acid moiety and/or a lipid comprising a C18:1 fatty acid moiety.

In embodiments, the present methods employ a single measurement to monitor GSL recovery, e.g., measuring the level of the at least one GSL at a single set point in time after challenge. In other embodiments, the monitoring involves monitoring GSL recovery over a period of time, e.g., measuring the level of the at least one GSL at more than one point in time after challenge. For example, monitoring the recovery of at least one glycosphingolipid following the challenge may comprise measuring the level of the at least one glycosphingolipid in the cell, or produced by the cell, a plurality of times following said challenge, e.g., 2, 3, 4, 5, 6, 7, 8, 9, 10, or more times. In embodiments, monitoring the recovery of at least one glycosphingolipid following the challenge comprises measuring the level of the at least one glycosphingolipid in the cell, or produced by the cell, at least 2, 3, 4, 5, 6, 7, 8, 9, or 10 times. In embodiments, monitoring the recovery of at least one glycosphingolipid following the challenge comprises measuring the level of the at least one glycosphingolipid in the cell, or produced by the cell, for as long as it takes to reach essentially the pre-treatment (baseline) level, e.g., a level which is within about 10% or within about 5% of the pre-treatment level. This provides information about the recovery of the GSL pathway, both in terms of the time taken to return to baseline GSL levels and also the profile of GSL recovery (e.g., the shape of the curve when concentration is plotted as a function of time).

As an alternative to (or in addition to) monitoring the recovery of the GSL pathway in the cell as compared to baseline levels from that cell, it is useful to compare the recovery of the GSL pathway in the subject's cells with the recovery in corresponding cells of a healthy individual. Such a comparison can help to identify aberrant GSL processing, especially where the aberration is not extremely severe. Thus, in embodiments the method further comprises a step of comparing the recovery of at least one glycosphingolipid following the challenge to the recovery of the same glycosphingolipid(s) following the same challenge to a corresponding cell from a healthy individual. In embodiments, the step of comparing involves comparing one or more of the following parameters (which may be derived, e.g., from a plot of concentration versus time): (i) maximum deviation of the concentration from baseline (Cₘₐₓ); (ii) time taken after challenge to reach Cₘₐₓ (Tₘₐₓ); (iii) area under the curve (AUC); and (iv) time taken for the level to return to baseline, e.g., to return to within about 10% or within about 5% of the baseline level. Using these metrics, the subject can be assessed as having aberrant glycosphingolipid processing if the difference in the parameter(s) is, for example, greater than about 10%, e.g., greater than about 20%, 30%, 50%, 75%, 100%, 150%, or 200%. In embodiments (e.g., where the sample is or comprises PBMCs obtained from the subject) the recovery of the glycosphingolipid(s) is monitored for a period of up to about 72 hours after challenge, e.g., for a period of up to about 64 hours, about 56 hours, about 48 hours, about 40 hours, about 32 hours, or about 24 hours after challenge.

Methods of the disclosure can also be used to assess disease state in an individual. Thus, in a second aspect the disclosure provides a method for determining the severity of a disease associated with aberrant glycosphingolipid processing in a subject. The method comprises assessing the subject for aberrant glycosphingolipid processing in accordance with the foregoing aspect and determining the severity of the disease based on the degree to which the recovery differs from that of a cell from a healthy individual which is challenged in the same way. The embodiments of the first aspect (e.g., concerning the agents to be used, sample types, and/or cell types, lipids to be monitored, etc.) can apply to this second aspect.

Diseases which are associated with aberrant glycosphingolipid processing include lysosomal storage diseases. Thus, this aspect may be particularly useful for assessing subjects who are known to have (or who are thought or predicted to have) a lysosomal storage disease. Such diseases typically involve alterations in GSL levels, often as a result of a deficiency in one or more enzymes associated with GSL processing. As demonstrated in the following Examples, a mouse model of Gaucher's disease (GD) demonstrates marked differences in lipid flux after CBE treatment as compared to wild-type mice. Thus, in embodiments the method may be used to determine the severity of a lysosomal storage disease in the subject, for example a disease selected from Fabry disease, Krabbe disease, Gaucher disease (e.g., type 1, 2, and 3), Niemann-Pick disease (e.g., type A, B, and C), metachromatic leukodystrophy, Farber disease, Krabbe disease, galactosialidosis, Schindler disease, GM1 gangliosidosis, GM2 gangliosidoses (e.g., AB variant, Sandhoff disease, and Tay-Sachs disease), Lysosomal acid lipase deficiency, Wolman disease, cholesteryl ester storage disease, multiple sulfatase deficiency, Pompe disease, Danon disease, Salla disease, alpha-mannosidosis, beta-mannosidosis, aspartylglucosaminuria, fucosidosis, MPS I (e.g., Hurler syndrome, Scheie syndrome, and Hurler-Scheie syndrome), MPS II (e.g., Hunter syndrome), MPS III (e.g., Sanfilippo syndrome type A, B, C, and D), MPS type IV (e.g., Morquio syndrome type A and B), MPS type VI (e.g., Maroteaux-Lamy syndrome), MPS type VII (e.g., Sly syndrome), MPS type IX (e.g., hyaluronidase deficiency), mucolipidosis (e.g., sialidosis, inclusion cell disease, pseudo-Hurler polydystrophy/phosphotransferase deficiency, and mucolipidin 1 deficiency), and neuronal ceroid lipofuscinoses (e.g., Santavuori-Haltia disease/infantile NCL, Jansky-Bielschowsky disease/late infantile NCL, Batten-Spielmeyer-Vogt disease/juvenile NCL, Kufs disease/adult NCL, Finnish variant/Type 5, Late infantile variant/type 6, type 7, Northern epilepsy/Turkish late infantile/type 8, German/Serbian late infantile/type 9, and Congenital cathepsin D deficiency). In one embodiment, the disease is GD (e.g., type 1, type 2, or type 3 GD). In another embodiment, the disease is FD.

Diseases which are associated with aberrant glycosphingolipid processing also include synucleinopathies. Thus, other conditions which may usefully be investigated using the methods of the disclosure include synucleinopathies. These are neurodegenerative diseases characterized by the abnormal accumulation of aggregates of alpha-synuclein protein in neurons, nerve fibres, or glial cells. There is, however, an emerging body of evidence that synucleinopathies can be associated with abnormal GSL processing. Thus, in embodiments, the method may be used to determine the severity of a synucleinopathy in the subject, for example a disease selected from Parkinson's disease (PD), e.g., idiopathic PD, or Dementia with Lewy Bodies (DLB). In embodiments, the disease is PD, e.g., idiopathic PD.

The present methods can be used to assess the disease state in individuals having known or unknown genotypes for genes that represent a risk factor for developing conditions associated with aberrant GSL processing (e.g., lysosomal storage diseases or synucleinopathies). In embodiments, the mutational status of the GBA gene in the subject is unknown; in other embodiments, the subject has been assessed as having only one or zero known mutant (e.g., non-functional or reduced function) GBA alleles (see e.g., Hruska et al., Hum. Mutat. (2008) 29 (5): 567-583). Likewise, the present methods can be used to assess the disease state in individuals whether or not they have previously been assessed for diseases associated with aberrant glycosphingolipid processing. Thus, in embodiments, the subject has not previously been diagnosed with a disease associated with aberrant glycosphingolipid processing (e.g., with a lysosomal storage disease such as Gaucher's disease, or with a synucleinopathy such as Parkinson's disease). In embodiments, the subject has not previously been diagnosed with Gaucher's disease. In embodiments, the subject has not previously been diagnosed with Parkinson's disease. In other embodiments, the subject has previously been diagnosed with a disease associated with aberrant glycosphingolipid processing and the method determines the progression of the disease.

In a third aspect the disclosure provides an in vitro method for diagnosing or monitoring the progression of a synucleinopathy (e.g., as defined herein) in a subject, the method comprising:
obtaining a sample from the subject comprising fibroblast cells, peripheral blood mononuclear cells, or induced pluripotent stem cells derived from somatic cells of the subject, and optionally culturing the cells;
contacting the sample with CBE;
measuring the level of glucosylceramide (GL1) and/or glucosylsphingosine (lyso-GL1) in or produced by the cells a plurality of times to obtain a response curve; and
comparing the response curve with a comparison response curve, wherein the comparison curve is either a standard response curve generated by challenging the same sample obtained from (a) a healthy individual or (b) a patient with a confirmed diagnosis of PD (to make a diagnosis of the subject), or the comparison curve is a response curve from a sample previously obtained from the same subject (to monitor the progression of the disease in the subject). It will be appreciated that embodiments of the first and second aspects (e.g., concerning the sample types and/or cell types, lipids to be monitored, disease state, etc.) apply to this third aspect.

In embodiments, the sample is contacted with CBE at a final concentration between about 0.01 μM and about 100 μM. For example, the final concentration of CBE may be between about 0.1 μM and about 10 μM, between about 0.3 μM and 8 μM, between about 0.5 μM and 5 μM, or between about 0.8 μM and 3 μM, e.g., about 1 μM or about 2 μM. In embodiments, an excipient such as sodium taurocholate is added to the sample to increase the dynamic range, e.g., by a factor of at least 2, at least 3, at least 4, or at least 5 times, e.g., by a factor of up to about 10 times.

As well as disease assessment, diagnosis, and monitoring, the present methods can also usefully be employed in assessing a therapeutic response to treatment, both before any treatment has been started (e.g., a predictive assessment) and also during a treatment regimen (e.g., monitoring a response). Such methods will typically assess the therapeutic response to treatment with a modulator of an enzyme involved in the glycosphingolipid pathway and will challenge the GSL pathway at or near the node represented by that enzyme. Glycosphingolipids which are closely related to that node (e.g., substrates or products of the enzyme) will typically be the ones that are monitored to assess recovery. Thus, for example, where an agent is used that is an inhibitor of glucosylceramide synthase (GCS) or an activator of glucocerebrosidase (GCase), the challenge can be to glucocerebrosidase (e.g., using CBE) and one or more of glucosylceramide (GL1), ceramide, and glucosylsphingosine (lyso-GL1) can be monitored.

Thus, in a fourth aspect the disclosure provides a method for assessing the likely therapeutic response of a subject to treatment with an inhibitor of glucosylceramide synthase (GCS) or an activator of glucocerebrosidase (GCase), the method comprising contacting a cell of the subject with CBE to challenge the glycosphingolipid pathway in the cell and monitoring the recovery of at least one glycosphingolipid following the challenge, whereby the subject is assessed as being a candidate for treatment if the recovery is slower or less complete than the recovery of a cell from a healthy individual which is challenged in the same way. It will be appreciated that embodiments of the foregoing aspects (e.g., concerning the sample types and/or cell types, lipids to be monitored, etc.) can apply to this fourth aspect.

In embodiments, the method assesses the likely therapeutic response of the subject to treatment with a GCS inhibitor (e.g., selected from eliglustat and venglustat, which includes the pharmaceutically acceptable salts thereof). In embodiments, the GCS inhibitor is venglustat or a pharmaceutically acceptable salt thereof. In other embodiments, the method assesses the likely therapeutic response of the subject to treatment with a GCase activator, for example selected from saposin C, ambroxol, N-(4-ethynylphenyl)-5,7-dimethylpyrazolo[1,5-a]pyrimidine-3-carboxamide, isofagomine, NN-DNJ, NCGC758, NCGC607, S-181, structurally targeted allosteric regulators (e.g., GT-02287 and GT-02329), and LTI-291.

In a fifth aspect the disclosure provides a method for assessing the therapeutic response of a subject to treatment with an inhibitor of glucosylceramide synthase (GCS) or an activator of glucocerebrosidase (GCase), the method comprising:
(a) obtaining a first cell-containing sample from the subject at a first time point;
(b) contacting a cell of the first cell-containing sample with CBE to challenge the glycosphingolipid pathway in the cell and monitoring the recovery of at least one glycosphingolipid following the challenge;
(c) obtaining a second cell-containing sample from the subject at a second time point after treatment of the subject with the inhibitor of GCS or activator of GCase; and
(d) contacting a cell of the second cell-containing sample with CBE to challenge the glycosphingolipid pathway in the cell and monitoring the recovery of at least one glycosphingolipid following the challenge,
whereby the treatment is assessed as being positive (e.g., successful) if the recovery in the cell from the second cell-containing sample is faster or more complete than the recovery in the cell from the first cell-containing sample. It will be appreciated that embodiments of the foregoing aspects (e.g., concerning the sample types and/or cell types, lipids to be monitored, etc.) can apply to this fifth aspect.

In embodiments, the steps (a) and (b) will be carried out in essentially the same way as the method steps (c) and (d). In this way, the difference between the GSL recovery in the cells from first and second samples may best reflect the efficacy of the treatment. Thus, typically the first cell-containing sample and the second cell-containing sample will comprise essentially the same cell types, e.g., they will be derived from the same type of tissue which has been handled and processed in essentially the same way. Likewise, it will typically be the case that the at least one glycosphingolipid which is monitored in step (b) will be the same as the at least one glycosphingolipid which is monitored in step (d).

The previous aspect also facilitates the optimization of a method of treatment in a subject, for example by providing a method of treating a disease associated with aberrant glycosphingolipid processing in a subject, the method comprising administering to the subject an agent which is capable of treating said disease, monitoring the therapeutic response to that agent, and modifying the dose of the agent accordingly.

Thus, in a sixth aspect the disclosure provides a method of treating a disease associated with aberrant glycosphingolipid processing (e.g., a lysosomal storage disease or a synucleinopathy as defined herein) in a subject in need thereof, the method comprising administering to the subject an agent which is capable of treating the lysosomal storage disease or the synucleinopathy, wherein the method involves monitoring the therapeutic response in accordance with the fifth aspect (above) and modifying the dose of the agent according to the result of the monitoring. It will be appreciated that embodiments of the foregoing aspects (e.g., concerning the sample types and/or cell types, lipids to be monitored, etc.) can apply to this sixth aspect.

In embodiments, the dose of the agent is kept the same if the treatment is assessed as being positive, and the dose of the agent is increased if the treatment is assessed as not being positive. In other embodiments, the dose of the agent is reduced if the treatment is assessed as being positive, with the monitoring steps being continued until the treatment is no longer assessed as being positive, at which time the dosage is increased until the treatment is assessed as being positive again. In embodiments, the monitoring steps are performed at regular intervals to assess the ongoing efficacy of the treatment and/or to allow for adjustment of the dosage so that the treatment may be assessed as being positive. The monitoring steps may be performed at intervals of, e.g., 1, 2, 3, 4, 5, 6, 12, 18, 24, or more months. In embodiments, the monitoring steps are performed more frequently at the start of treatment (e.g., every 1, 2, 3, or 4 weeks) and are then performed less frequently (e.g., every 2, 3, 4, 6, or 12 months) once a stable treatment regimen has been established.

In embodiments, the monitoring steps are performed alongside other known methods of monitoring the progress of diseases associated with aberrant glycosphingolipid processing, e.g., lysosomal storage diseases. For example, the measuring steps may be conducted alongside measurement of the subject's platelet count, hemoglobin concentration, spleen volume, and/or liver volume. These measurements can be useful, e.g., in determining whether the subject is experiencing more severe symptoms of a lysosomal storage disease.

In a seventh aspect, the disclosure provides a method of treating a lysosomal storage disease or a synucleinopathy in a subject in need thereof, the method comprising the step of administering an effective amount of an agent which is capable of treating the lysosomal storage disease or the synucleinopathy (e.g., an agent as described herein), wherein the patient has been assessed as having aberrant glycosphingolipid processing according to a method as described herein, or has had their disease state or severity assessed according to a method as described herein.

In an eighth aspect, the disclosure provides a method of treating or preventing the development or progression of a lysosomal storage disease or a synucleinopathy in a subject assessed as being at risk of developing a lysosomal storage disease or a synucleinopathy according to a method as described herein (e.g., the method of the second aspect or the third aspect), the method comprising the steps of: (a) starting the subject on a course of therapeutic treatment (e.g., a therapeutically effective amount of an agent which is capable of treating the lysosomal storage disease or the synucleinopathy, such as an agent as described herein); and optionally (b) assessing or repeating the assessment of risk of developing a lysosomal storage disease or a synucleinopathy according to a method as described herein, and optionally adjusting the therapeutic treatment based on the new assessment.

In embodiments, the method comprises the administration of one or more (e.g., two, three, four, or more) agents capable of treating or preventing the disease or disorder. For example, the method may comprise administration of a lysosomal enzyme (ERT) such as imiglucerase and/or a small molecule (SRT) such as e.g., venglustat or eliglustat. Alternatively, or in addition, the method may comprise administration of one or more agents which mitigate the symptoms of the disease or disorder, e.g., selected from a levodopa or a prodrug thereof (optionally in combination with carbidopa or a prodrug thereof), ambroxol, amantadine, a dopamine agonist, a MAO inhibitor, a COMT inhibitor, and an anticholinergic agent.

In a ninth aspect, the disclosure provides the use of conduritol-β epoxide (CBE) in a method of diagnosing or determining the severity of a disease associated with aberrant glycosphingolipid processing in a subject. It will be appreciated that embodiments of the foregoing aspects (e.g., concerning the disease) can apply to this ninth aspect.

Having been generally described herein, the follow non-limiting examples are provided to further illustrate this disclosure.

### EXAMPLES

### Example 1: In Vivo Proof of Concept Studies in Mice

This example aimed to test the hypothesis that transient inhibition of GCase and the subsequent accumulation of GCase substrates could serve as a sensitive assay for uncovering defects in lipid flux. Initial proof of concept studies were carried out in mice and transformed human cell lines.

### Dose Response of GSL Pathway Challenge in WT Mice

A dose response to CBE was performed in wild-type (WT) mice (Charles River Laboratories or Jackson Labs). Concentrations between 0.3 mg/kg and 100 mg/kg of CBE were investigated. GCase activity remaining 24 hours after administration of CBE was measured using a 4-methylumbelliferone (4MU) hydrolysis assay. Briefly, cell lysates from different sample types were diluted into 0.1 M sodium acetate buffer, pH 4.5 containing 10 mM of synthetic substrate 4-methylumbelliferyl-β-D-glucopyranoside and incubated at 37° C. for 1 hour. The reaction was terminated by adding 0.5 volume of 1 M glycine buffer, pH 12.5. The fluorescence of the reaction was measured using a Spectramax fluorimeter (Ex365/Em445; Molecular Devices, Sunnyvale, CA, USA). A standard curve was generated using Cerezyme® (imiglucerase).

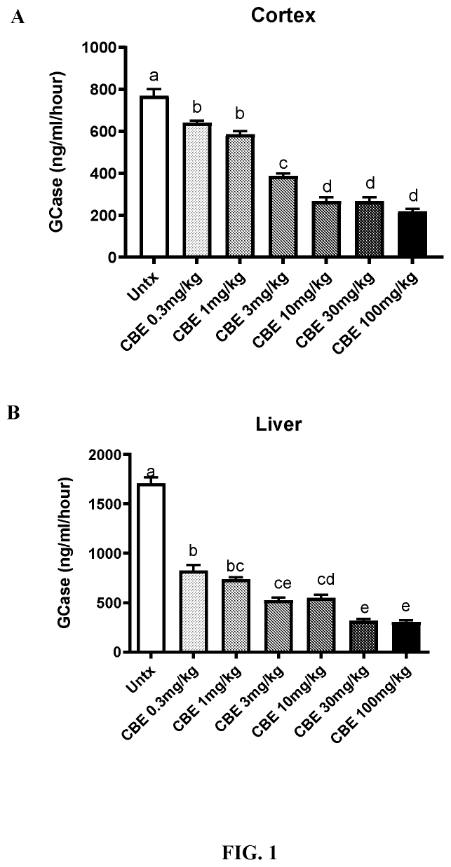

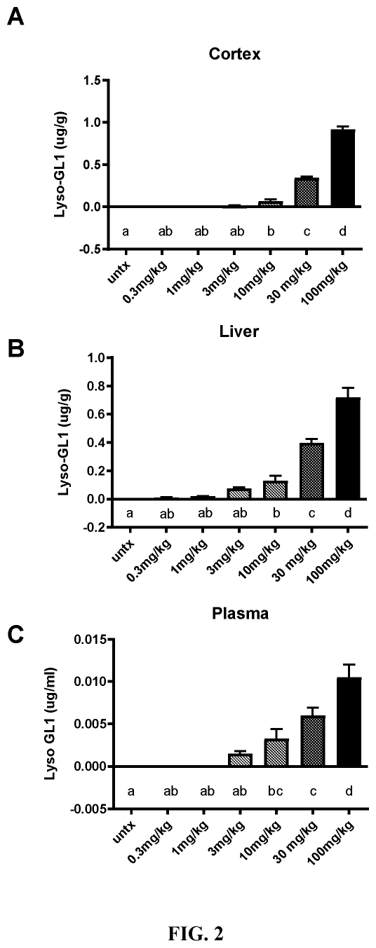

FIG. 1 shows the results of CBE administration in WT mice. Although there was a dose-dependent reduction in enzyme activity, little or no further inhibition of GCase was measured beyond 30 mg/kg. With only these data, it was difficult to determine whether this is due to saturation of the enzyme or due to the sensitivity of the GCase activity assay. Measurement of downstream substrates allowed for a discrimination between these two cases. Lyso-GL1 levels in cortex, liver, and plasma all increased with increasing CBE dose beyond 30 mg/kg (FIG. 2), suggesting that this substrate can act as a reporter for GCase activity across a wider range of concentrations.

Timecourse of Lipid Flux after GSL Pathway Challenge in Gba+/− Mice

It was investigated whether substrate accumulation could allow for discrimination between mice harboring different copy numbers of mutations in GBA1. Three mouse models were investigated: Gba^{D409V/D409V}, Gba^{D409V/+}, and Gba^{+/+}, which are homozygous for a loss of function D409V mutation in GBA1, heterozygous for that mutation, or WT for GBA1, respectively. The GCase substrate, glucosylceramide (GL1) was investigated in addition to glucosylsphingosine (lyso-GL1). Mice were administered a single dose of CBE and sacrificed after 3, 6, 24, 48, or 72 hours. Substrate accumulation and dissipation was measured over time.

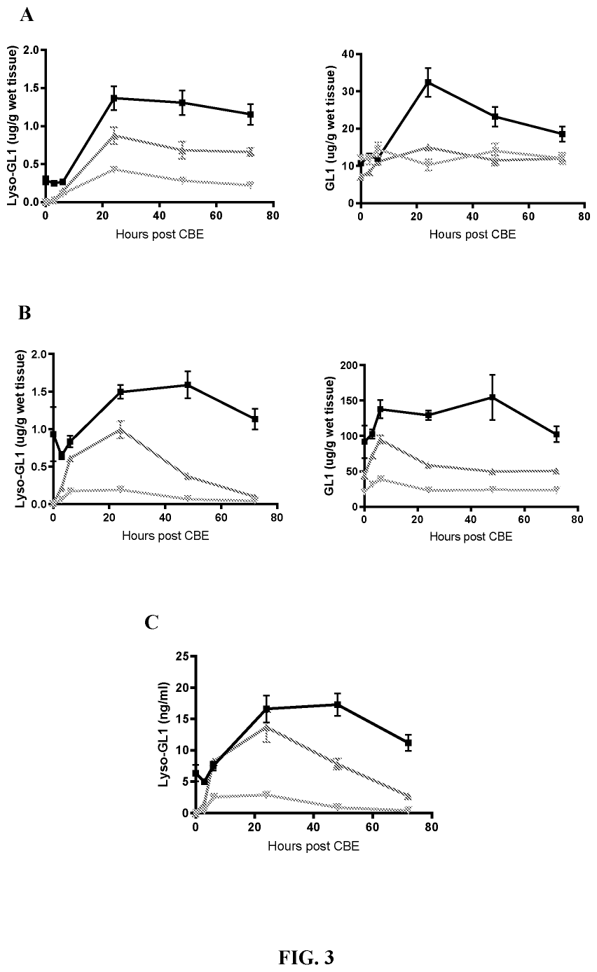

Between 24-48 hours, the substrate accumulation peaked, with Lyso-GL1 accumulation in the cortex, liver, and plasma showing greater sensitivity to the genetic background (FIG. 3). After the peak, the substrate levels began to drop as newly synthesized enzyme degraded these substrates. It is possible that secondary pathways were also contributing to the lipid reductions observed. As predicted, the peak lipid concentration inversely correlated with the genotype. In addition, the rate of recovery correlated with genotype: the WT mice restored or approached normal levels the fastest, followed by the heterozygous mutant mice, followed by the homozygous mutant mice.

Timecourse of Lipid Flux after GSL Pathway Challenge in Wild-Type Mice

Having demonstrated that accumulation and dissipation of substrates was a sensitive measure of the amount of GCase activity, the next question tested was how the levels of substrates change over time after challenge in wild-type mice.

Three different wild-type mouse strains (C57BL/6J, BALB/cJ, and FVB-Charles River Laboratories or Jackson Labs) were evaluated for GSL accumulation and restoration after CBE challenge. These strains are all “normal”, in the sense that they do not harbor any particular mutations but are genetically different from each other just as individual people are. Importantly, the chosen mice do not have mutations in GBA1. The CBE challenge time course was performed in these three different strains, measuring lyso-GL1 and GL-1 accumulation at 24, 48, and 72 hours after administration.

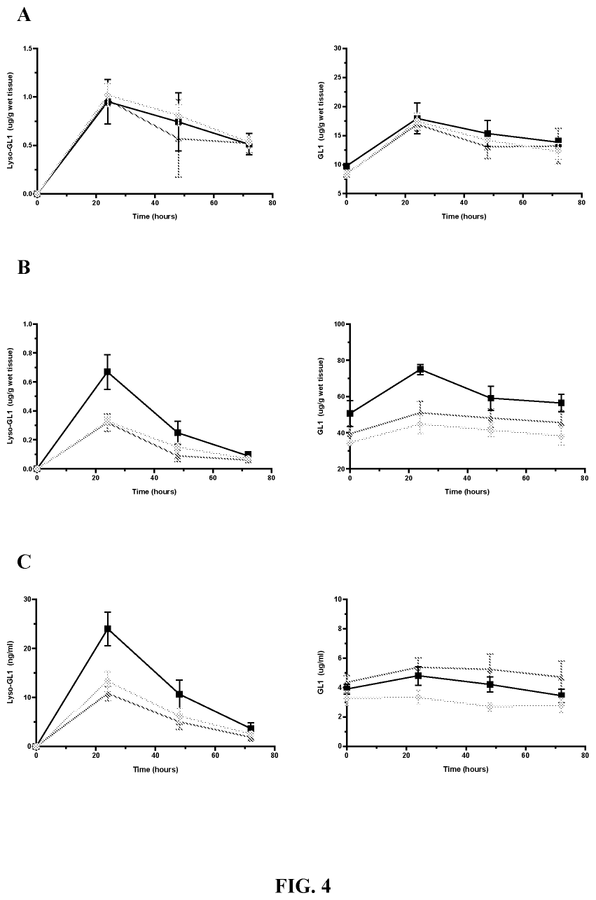

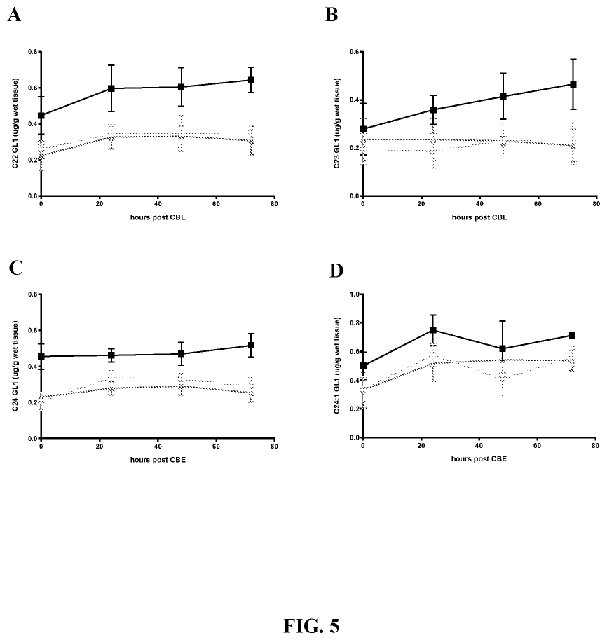

Results of the experiment are shown in FIG. 4 and FIG. 5. There was no significant difference between the strains in GSL levels in the cortex (FIG. 4A). Yet, in the liver and in plasma, the C57BL/6 mice accumulated much more lyso-GL1 than the BALB/cJ and FVB strains (FIGS. 4B and 4C). These results were mirrored for total GL1 in the liver as well. Plasma GL1 levels show a slightly different trend, with the highest levels observed for FVB mice. These results indicate that the CBE challenge has the potential to discriminate between individuals who might harbor deficiencies in their sphingolipid metabolism pathway, even if they do not harbor GBA1 mutations. Analysis of different isoforms of GL1 in cortex showed a variable distribution of chain lengths between strains (FIG. 5), with C57BL/6 mice having higher levels of C22, C23, C24, and C24:1 isoforms.

### Example 2: In Vitro Studies in Human Cell Lines

The above in vivo experiments provide a proof-of-concept for transiently inhibiting GCase to increase the accumulation of sphingolipids, thereby amplifying the signal from underlying genetic differences. Such methods are, however, impractical for use in patients because of the tissue samples that must be extracted to make the measurements. To bridge this gap in feasibility, human cell lines with different levels of GBA1 transcript expression were investigated.

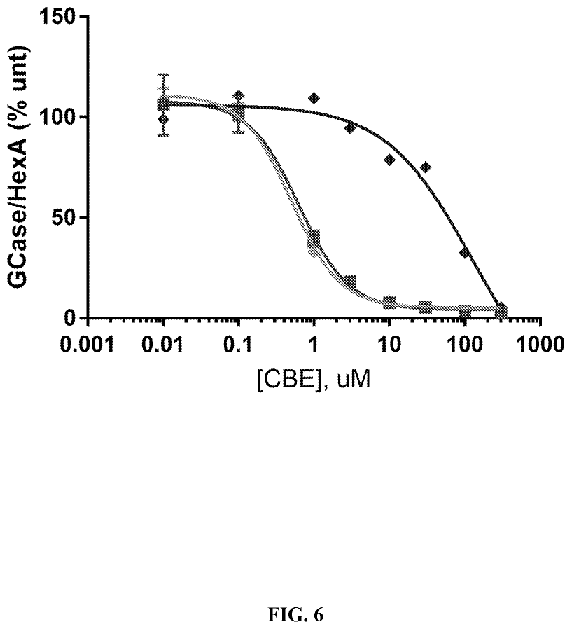

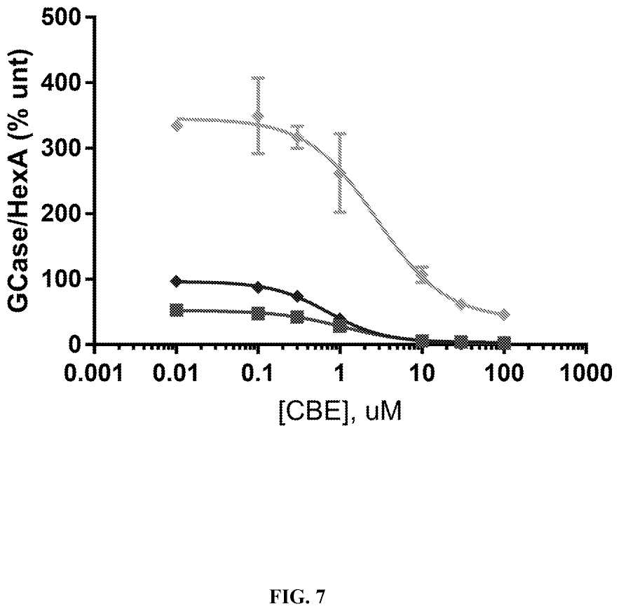

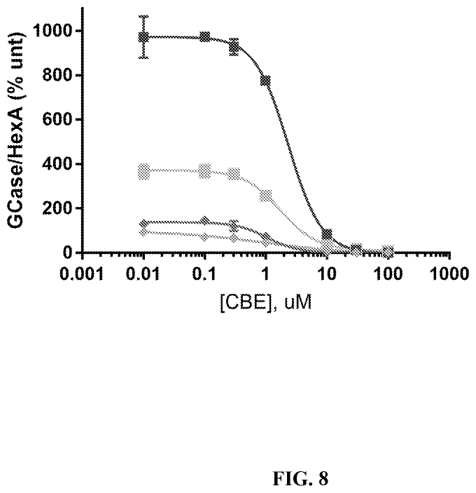

First, the response of human SHSY-5Y neuroblastoma cells to CBE was studied. Cells were either used without further changes (wild-type) or were modified to reduce or increase Gba activity (knock-down and over-expressed, respectively). The modification was effected by stable transfection with a plasmid expressing an siRNA targeting GBA (KD) or a plasmid constitutively overexpressing GBA (OE). Human SHSY-5Y neuroblastoma cells (Sigma) were incubated with 100 μM CBE (Toronto Research Chemicals) for 24 hours. Enzyme activity and lipid levels were measured at −24 hours (before CBE incubation), and at 0, 24, and 48 hours after CBE removal. FIG. 6 shows the impact on enzyme activity of different durations of incubation of the wild-type cells with CBE, from which it was concluded that a 24-hour incubation period was suitable to get a good response. FIG. 7 shows the response of the three different cell types to various concentrations of CBE. A marked increase in enzyme activity was observed between the knock-down and wild-type cells, and between the wild-type and over-expressed cells. Interestingly, the dynamic range of the assay was found to be significantly increased when the cells were also incubated with sodium taurocholate (FIG. 8).

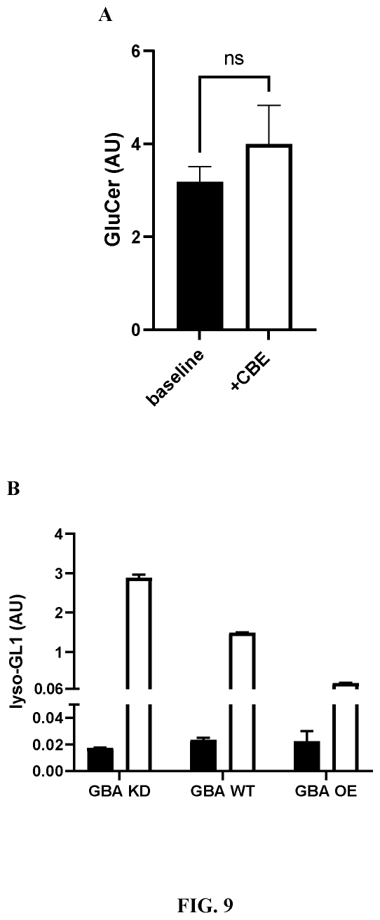

The lipid response of cells to CBE incubation was then investigated (FIG. 9). No significant difference in total GL1 levels was observed in wild-type cells after treatment (FIG. 9A). While some differences could be measured in lyso-GL1 levels after CBE treatment between the three cell types (FIG. 9B), there was no measurable accumulation of lyso-GL1 in the absence of treatment. Under typical cell culture conditions, it was found not to be possible to measure significant differences in GL1 accumulation between cells expressing 40% of endogenous GBA1, the WT amount, or 300% the WT amount.

Furthermore, none of these cell lines had a measurable accumulation of lyso-GL1 under normal conditions. The consequences of the genetic differences between the cells could only be revealed by the transient inhibition of GCase with CBE. Upon CBE treatment, the cells accumulated GL1 and lyso-GL1 in levels inversely proportional to the level of GBA1 expression. This model in vitro system suggests that cells harvested from patients and treated with CBE in culture can be used to measure inherent deficiencies in the patients' sphingolipid metabolism pathway.

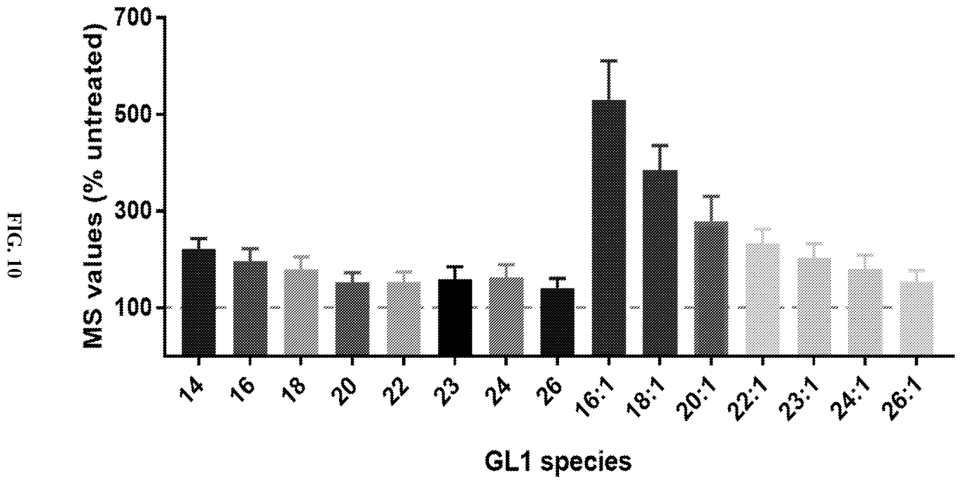

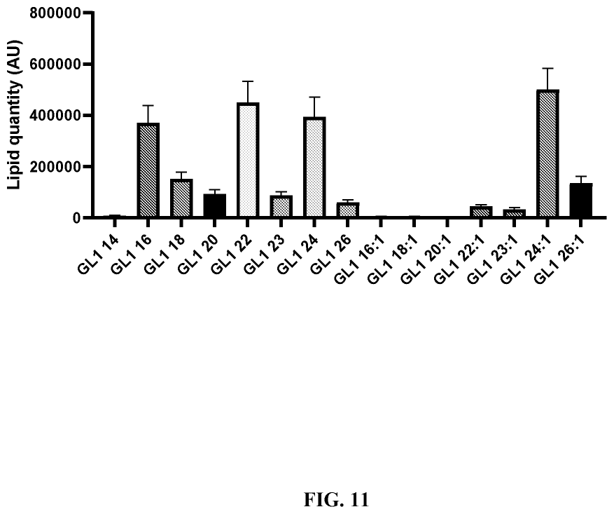

The accumulation of different GL1 species after CBE treatment was also investigated. Generally, it was found that mono-unsaturated GL1 species (i.e., isoforms having one double bond in the fatty acid portion of the molecule) accumulated more than saturated species, as did isoforms having a shorter chain length (FIG. 10). Considering the baseline abundance of GL1 species (FIG. 11), it was observed that the low abundance isoforms were generally the ones to accumulate in response to CBE treatment.

### Example 3: CBE Challenge in Human PBMCs

The impact of CBE challenge in human peripheral blood mononuclear cells (PBMCs) was investigated.

PBMCs were obtained from blood samples (Stem Cell Technologies, catalog number 70025, lot number 2105417005, Donor ID CE0006419) using standard procedures. 25,000 cells were added to individual wells of a multi-well plate and allowed to acclimate for 24 hours before they were treated with 100 μM CBE for 24 hours. Culture medium was removed and replaced with fresh medium (without CBE), and lipids were extracted from the cells at time intervals of 0, 2, 4, 8, 16 and 24 hours. GL1 and lyso-GL1 levels were measured, as were levels of phosphatidylcholine (PC). PC levels were found to be largely unaffected by CBE treatment and so were used to normalize the levels of GL1 and lyso-GL1 (e.g., to minimize the impact of different numbers of cells in each well, and/or the effects of cell proliferation during the timecourse).

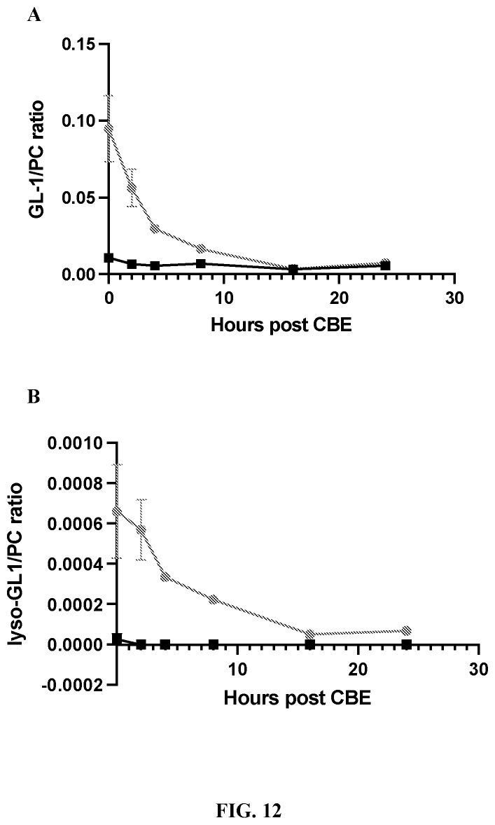

GL1 levels in the PBMCs increased about 6-fold after CBE treatment (from about 2 ng/ml to about 12 ng/ml), and lyso-GL1 levels increased from nearly undetectable levels to about 0.08 ng/ml. The lyso-GL1 levels were around the lower limit of detection of the assay, and so were subject to larger errors. When normalized to total PC levels (100-600 ng/ml over the course of the experiments), the GL1 and lyso-GL1 levels were as shown in FIG. 12. GL1 levels (FIG. 12A) and lyso-GL1 levels (FIG. 12B) had normalized 16 hours after treatment.

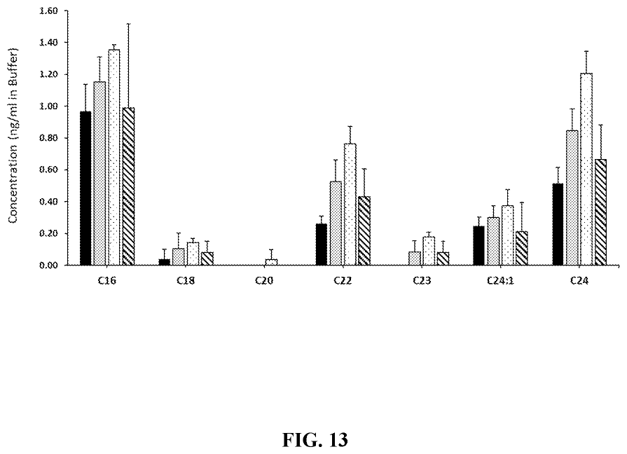

An analysis of GL1 isoforms was performed to check the stability of the cell cultures. Cells were obtained and cultured as described above without CBE treatment and were monitored for 72 hours. Lipid levels of GL1 isoforms are shown in FIG. 13. Although the different isoforms display different absolute levels, there is a pattern to all species tested, namely that levels increase in a roughly linear fashion from 0 to 24 to 48 hours, and then drop at 72 hours. It is postulated that the increase in levels to 48 hours is due to cell proliferation, whereas the reduction at 72 hours may be due to heterogenous cell population growth and/or adherence dynamics. These results suggest that a timecourse for analysis of GSL levels in PBMCs using this protocol should typically not proceed beyond 48 hours after challenge.

In summary, PBMCs are a suitable cell type for monitoring GSL flux after challenge—a timecourse can be established from cells obtained from around 1 ml of whole blood. Normalization of GSL levels to PC concentration can reduce systematic errors. PC is not integral to the GSL pathway, and its concentration is not affected by CBE treatment. GL1 appears to report better on CBE challenge and recovery than lyso-GL1. There is a robust increase in GL1 levels after challenge which returns to baseline by 16 hours in healthy individuals.

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method of assessing a subject for aberrant glycosphingolipid processing, the method comprising treating a cell of the subject to challenge the glycosphingolipid pathway in the cell and monitoring the recovery of at least one glycosphingolipid following the challenge.

2. The method of claim 1, wherein the challenge comprises contacting the cell with an agent which inhibits an enzyme involved in the glycosphingolipid pathway.

3. The method of claim 2, wherein the enzyme is glucocerebrosidase.

4. The method of claim 2 or 3, wherein the agent is selective for one enzyme involved in the glycosphingolipid pathway.

5. The method of any one of claims 2 to 4, wherein the agent is an irreversible inhibitor of the enzyme, e.g., wherein the inhibitor binds covalently to the enzyme.

6. The method of any one of claims 2 to 5, wherein the agent is a compound of formula (I): 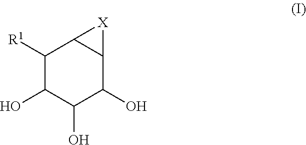
or a pharmaceutically acceptable salt thereof, wherein:
X is —O— or —N(R²)—;
R¹ is selected from —OH, —C(O)OH and —CH₂OH; and
R², when present, is selected from —H and C₁₋₆-alkyl optionally substituted by one or more groups independently selected from —OH and halogen.

7. The method of claim 1, wherein the step of treating a cell of the subject to challenge the glycosphingolipid pathway in the cell comprises contacting the cell with conduritol-β epoxide (CBE).

8. The method of any one of claims 1 to 7, wherein the method is an in vitro method in which the cell is present in a sample obtained from the subject.

9. The method of claim 8, wherein the sample is a tissue sample (e.g., from the brain, liver, kidney, skin, or spleen of the subject) or wherein the sample is a cell-containing blood sample (e.g., whole blood).

10. The method of any one of claims 1 to 9, wherein the cell is a fibroblast, a peripheral blood mononuclear cell (PMBC), or an induced pluripotent stem cell (iPSC) derived from a somatic cell of the subject.

11. The method of claim 10, wherein the cell is a peripheral blood mononuclear cell which has been obtained (e.g., purified) from a blood sample taken from the subject.

12. The method of any one of claims 1 to 11, wherein the cell expresses neuroepithelial stem cell protein (nestin).

13. The method of any one of claims 1 to 12, wherein monitoring the recovery of at least one glycosphingolipid following the challenge comprises measuring the level of at least one glycosphingolipid in the cell, or produced by the cell, a plurality of times following the challenge, e.g., 2, 3, 4, 5, 6, 7, 8, 9, 10, or more times.

14. The method of any one of claims 1 to 13, wherein monitoring the recovery of at least one glycosphingolipid following the challenge comprises measuring the level of at least one glycosphingolipid in the cell, or produced by the cell, for as long as it takes to reach essentially the pre-treatment (baseline) level, e.g., a level which is within about 10% or within about 5% of the pre-treatment level.

15. The method of any one of claims 1 to 14, wherein the at least one glycosphingolipid is or comprises a lipid selected from glucosylceramide (GL1), ceramide, and glucosylsphingosine (lyso-GL1).

16. The method of claim 15, wherein the at least one glycosphingolipid is or comprises total GL1, total ceramide, or total lyso-GL1.

17. The method of any one of claims 1 to 16, wherein the at least one glycosphingolipid is or comprises a lipid comprising a monounsaturated fatty acid moiety, e.g., selected from C16:1, C18:1, C20:1, C22:1, or C24:1.

18. The method of any one of claims 1 to 16, wherein the at least one glycosphingolipid is or comprises a lipid comprising a fatty acid moiety selected from C24:0, C16:1 n-7, C18:1 n-7, C22:1 n-9, C24:1 n-9, and C18:2 cis.

19. The method of any one of claims 1 to 18, wherein monitoring the recovery of at least one glycosphingolipid following the challenge does not comprise monitoring the recovery of a lipid comprising a C18:0 fatty acid moiety and/or a lipid comprising a C18:1 fatty acid moiety.

20. The method of any one of claims 1 to 19, further comprising a step of comparing the recovery of the at least one glycosphingolipid following the challenge to the recovery of the same glycosphingolipid(s) following the same challenge to a corresponding cell from a healthy individual.

21. The method of claim 20, wherein the step of comparing involves comparing one or more of the following parameters (which may be derived from a plot of concentration versus time): (i) maximum deviation of the concentration from baseline (Cₘₐₓ); (ii) time taken after challenge to reach Cₘₐₓ (Tₘₐₓ); (iii) area under the curve (AUC); and (iv) time taken for the level to return to baseline, e.g., to return to within about 10% or within about 5% of the baseline level.

22. The method of claim 21, wherein the subject is assessed as having aberrant glycosphingolipid processing if the difference in the parameter(s) is greater than about 10%, e.g., greater than about 20%, 30%, 50%, 75%, 100%, 150%, or 200%.

23. A method for determining the severity of a disease associated with aberrant glycosphingolipid processing in a subject, the method comprising assessing the subject for aberrant glycosphingolipid processing in accordance with any one of claims 1 to 22 and determining the severity of the disease based on the degree to which the recovery differs from that of a cell from a healthy individual which is challenged in the same way.

24. The method of claim 23, wherein the disease is a lysosomal storage disease, e.g., Gaucher's disease.

25. The method of claim 23, wherein the disease is a synucleinopathy such as Parkinson's disease (PD), e.g., idiopathic PD.

26. The method of any one of claims 23 to 25, wherein the mutational status of the GBA gene in the subject is unknown, or wherein the subject has been assessed as having only one or zero known mutant (e.g., non-functional or reduced function) GBA alleles.

27. The method of any one of claims 23 to 26, wherein the subject has not previously been diagnosed with a disease associated with aberrant glycosphingolipid processing, e.g., with a lysosomal storage disease such as Gaucher's disease or with Parkinson's disease.

28. The method of any one of claims 23 to 26, wherein the subject has previously been diagnosed with a disease associated with aberrant glycosphingolipid processing and the method determines the progression of the disease.

29. An in vitro method for diagnosing or monitoring the progression of a synucleinopathy such as Parkinson's disease (PD), e.g., idiopathic PD, in a subject, the method comprising:
obtaining a sample from the subject comprising fibroblast cells, peripheral blood mononuclear cells, or induced pluripotent stem cells derived from somatic cells of the subject, and optionally culturing the cells;
contacting the sample with CBE;
measuring the level of glucosylceramide (GL1) and/or glucosylsphingosine (lyso-GL1) in or produced by the cells a plurality of times to obtain a response curve; and
comparing the response curve with a comparison response curve, wherein the comparison curve is either a standard response curve generated by challenging the same sample obtained from (a) a healthy individual or (b) a patient with a confirmed diagnosis of PD (to make a diagnosis of the subject), or the comparison curve is a response curve from a sample previously obtained from the same subject (to monitor the progression of the disease in the subject).

30. A method for assessing the likely therapeutic response of a subject to treatment with an inhibitor of glucosylceramide synthase (GCS) or an activator of glucocerebrosidase, the method comprising contacting a cell of the subject with CBE to challenge the glycosphingolipid pathway in the cell and monitoring the recovery of at least one glycosphingolipid following the challenge, whereby the subject is assessed as being a candidate for treatment if the recovery is slower or less complete than the recovery of a cell from a healthy individual which is challenged in the same way.

31. The method of claim 30, wherein the GCS inhibitor is venglustat or a pharmaceutically acceptable salt thereof.

32. The method of claim 30 or 31, wherein the cell is a fibroblast, a peripheral blood mononuclear cell, or an induced pluripotent stem cell derived from a somatic cell of the subject.

33. The method of claim 32, wherein the cell is a peripheral blood mononuclear cell which has been obtained (e.g., purified) from a blood sample taken from the subject.

34. Use of conduritol-ß epoxide in a method of diagnosing or determining the severity of a disease associated with aberrant glycosphingolipid processing in a subject.

35. The use of claim 34, wherein the disease is a lysosomal storage (e.g., Gaucher's disease) or a synucleinopathy such as Parkinson's disease (e.g., idiopathic PD).

36. A method for assessing the therapeutic response of a subject to treatment with an inhibitor of glucosylceramide synthase (GCS) or an activator of glucocerebrosidase (GCase), the method comprising:
(a) obtaining a first cell-containing sample from the subject at a first time point;
(b) contacting a cell of the first cell-containing sample with CBE to challenge the glycosphingolipid pathway in the cell and monitoring the recovery of at least one glycosphingolipid following the challenge;
(c) obtaining a second cell-containing sample from the subject at a second time point after treatment of the subject with the inhibitor of GCS or activator of GCase; and
(d) contacting a cell of the second cell-containing sample with CBE to challenge the glycosphingolipid pathway in the cell and monitoring the recovery of at least one glycosphingolipid following the challenge,
whereby the treatment is assessed as being positive if the recovery in the cell from the second cell-containing sample is faster or more complete than the recovery in the cell from the first cell-containing sample.

37. The method of claim 36, wherein the treatment of the subject is treatment with the GCS inhibitor venglustat, or a pharmaceutically acceptable salt thereof.

38. The method of claim 36 or 37, wherein the cell of the first cell-containing sample and/or the cell of the second cell-containing sample is independently selected from a fibroblast, a peripheral blood mononuclear cell, or an induced pluripotent stem cell derived from a somatic cell of the subject.

39. The method of any one of claims 36 to 38 wherein: the steps (a) and (b) are carried out in essentially the same way as the method steps (c) and (d); the first cell-containing sample and the second cell-containing sample comprise essentially the same cell types; and/or the at least one glycosphingolipid which is monitored in step (b) is the same as the at least one glycosphingolipid which is monitored in step (d).

40. A method of treating a lysosomal storage disease or a synucleinopathy in a subject in need thereof, the method comprising administering to the subject an agent which is capable of treating the lysosomal storage disease or the synucleinopathy, wherein the method involves monitoring the therapeutic response in accordance with any one of claims 36 to 39, and modifying the dose of the agent according to the result of the monitoring.

41. The method of claim 40, wherein the dose of the agent is kept the same if the treatment is assessed as being positive and the dose of the agent is increased if the treatment is assessed as not being positive; or wherein the dose of the agent is reduced if the treatment is assessed as being positive, with the monitoring steps being continued until the treatment is no longer assessed as being positive, at which time the dosage is increased until the treatment is assessed as being positive again.

42. A method of treating a lysosomal storage disease or a synucleinopathy in a subject in need thereof, the method comprising the step of administering an effective amount of an agent which is capable of treating the lysosomal storage disease or the synucleinopathy, wherein the subject has been assessed as having aberrant glycosphingolipid processing according to the method of any one of claims 1 to 22, or has had their disease state or severity assessed according to the method of any one of claims 23 to 29.

43. A method of treating or preventing the development or progression of a lysosomal storage disease or a synucleinopathy in a subject assessed as being at risk of developing a lysosomal storage disease or a synucleinopathy according to the method of any one of claims 23 to 29, the method comprising the steps of:
(a) starting the subject on a course of therapeutic treatment; and optionally
(b) assessing or repeating the assessment of risk of developing a lysosomal storage disease according to the method of any one of claims 23 to 29, and optionally adjusting the therapeutic treatment based on the new assessment.
